(19) United States
(12) Patent Application Publication
(10) Pub. No.: US 2022/0096697 A1
(43) Pub. Date: Mar. 31, 2022

(54) SYSTEM FOR SCENTING ROOMS

(71) Applicant: STEARINOS OOD, Silistra (BG)

(72) Inventor: Herbert Wittekind, Silistra (BG)

(21) Appl. No.: 17/481,997

(22) Filed: Sep. 22, 2021

(30) Foreign Application Priority Data
Sep. 30, 2020 (EP) 20199176.7

(51) Int. Cl. A61L 9/03

(52) U.S. Cl. CPC A61L 9/037; A61L 2209/134

(57) ABSTRACT

A sustainable system for scenting rooms, having a solid body for the situation and evaporation of a liquid mixture that contains at least one fragrance, the body having a porous, ceramic, and reusable material and the material of the body having an open porosity that is greater than 50%, the mixture having a flash point that is equal to or below 120° C. In addition, a method is described for producing the body and for preparing the system for reuse.

### CROSS-REFERENCES TO RELATED APPLICATIONS

This application claims the benefit of the European patent application No. 20199176.7 filed on Sep. 30, 2020, the entire disclosures of which are incorporated herein by way of reference.

### FIELD OF THE INVENTION

The present invention relates to a system for scenting rooms. The system includes a body that contains a liquid mixture having at least one fragrance, and the mixture. Furthermore, a method for manufacturing such a body is described, as well as a mixture dispenser.

### BACKGROUND OF THE INVENTION

It has been common practice for a long time to add odorous substances (odorants, fragrances) to the air. The targeted scenting of rooms serves to make the stay in the respective room more pleasant for the persons in the room, because any unpleasant odors are concealed or because a certain positive feeling (e.g., a recognition effect) can be triggered by the fragrances, for example in hotels or supermarkets. The use of fragrances is based on the observation that, for humans, smelling is an important sensory impression, alongside seeing, hearing or touching. In this context, smell (i.e., olfactory perception) is the interpretation of sensory excitations transmitted to the brain by the chemoreceptors of the nose or other olfactory organs. According to a recent study, humans are thought to be able to distinguish over one trillion odors.

Various systems are known for the introduction of fragrances into the air. A simple and widespread possibility is the use of scented candles. However, scented candles burn quickly and are not suitable for permanent use. The flame is also a fire hazard.

From EP 3 488 872 A1, a fragrance dispenser having a fragrance reservoir is known, which is accommodated in a housing, the housing being formed as an in-wall housing. The fragrance may be accommodated in a fragrance reservoir made up of a porous, air-permeable molded body made of a multiplicity of plastic particles, the plastic particles comprising fragrances. If such a fragrance dispenser is to be used over a long period of time, the fragrance reservoir (i.e., the molded body having the fragrance plastic particles) must be replaced by a new fragrance reservoir after a certain period of time. The used fragrance reservoir is disposed of.

Currently, spray systems with spray cans are frequently used for scenting rooms, which release a spray of fragrance at regular time intervals. For example, spray cans with a capacity of 100 ml to 150 ml are used, which can scent a room for about 4 to 6 weeks. Afterwards, the empty spray can is disposed of. This generates much waste.

At present and in the future, environmental protection is becoming increasingly important. Against this background, solutions have been sought as to how a system for scenting rooms can be designed in a more resource-efficient way. At the same time, a system for scenting rooms is to be provided that has a long effective time, in order to minimize the effort involved in using such a system. At the same time, it should be possible to change fragrances in order to enable the use of a new fragrance after a certain period of time, since after a certain period of time a fragrance is no longer perceived at all or is perceived at a lower strength.

### SUMMARY OF THE INVENTION

The above problem is solved, in particular, by a system for scenting rooms comprising a solid body that accommodates a liquid mixture and the liquid mixture, which contains at least one fragrance, the body having a porous, ceramic and reusable material having an open porosity greater than 50%. According to the present invention, the mixture has a flash point equal to or lower than 120° C.

The system according to the present invention is made up of the solid, self-supporting, ceramic, porous body, used to contain and evaporate the initially liquid mixture, and the mixture itself. The mixture, with its at least one fragrance for scenting the room, gradually transitions to a gaseous state by evaporation and escapes from the porous body over a predetermined period of time. The open porosity, which is greater than 50%, preferably greater than 80%, particularly preferably greater than 90%, enables the mixture to be easily absorbed or, as desired, easily released via evaporation into or out of the body. Here, open porosity is defined as the portion of the cavities of the body that are in communication with each other or with the environment. In contrast, cavities form a closed porosity if they are closed off from each other or from the environment. Here, the above statements refer to the volume of the pores, namely the volume of the open pores in relation to the volume of all pores (open porosity) or the volume of closed pores in relation to the volume of all pores. The porosity can be determined by, for example, mercury porosimetry.

In an exemplary embodiment, the ceramic material exhibits a bimodal pore size distribution when the frequency of occurrence of pores is plotted with respect to their size. Here, pore size is understood as the inner diameter of the cavity (pore). This means that most pores of the ceramic material can be assigned to a first group with a smaller pore diameter or to a second group with a larger pore diameter. This pore size distribution has the effect that the dispensing rate of the mixture is within the desired range. For example, 50 g of mixture can scent a room of a size of 20 m2 for a period in the range of 1 week to 6 months. In addition, the mixture is fully absorbed into the body so that the mixture does not come out when the body is touched.

In an exemplary embodiment, the pore size of the first group of pores (i.e., a first maximum of the bimodal pore size distribution) is in the range between 1 mm and 3 mm, preferably between 2 mm and 3 mm, and the pore size of the second group of pores (i.e., a second maximum of the pore size distribution) is in the range between 0.01 mm and 0.3 mm, preferably between 0.05 mm and 0.3 mm. The combination according to the present invention of large and small pores (together with the mixture adapted for this and described below) results in a very high absorption capacity of the body for the liquid containing at least one fragrance. In order to achieve comparability of the bodies when different substances are used, the absorption capacity of the body is measured using water. In a body according to the present invention, the water absorption is at least 70% of the weight of the body, preferably at least 80% of the weight of the body, particularly preferably at least 90% of the weight of the body. This means, for example, that if the body has a weight of 300 g, the absorption capacity of water is at least 210 g, preferably at least 240 g, particularly preferably at least 270 g. The high absorption capacity of the porous body brings it about that a long scenting time can be realized without the need for maintenance and refilling.

In another exemplary embodiment, the body predominantly has pores with a small pore diameter. This pore size causes the delivery rate of the fragrance to be within the desired range. For example, 50 g of a liquid mixture containing at least fragrance can scent a room having a size of 20 m2 over a period of time in the range of 1 week to 5 months. Moreover, the mixture is completely absorbed into the body, so that the mixture does not come out when the body is touched. The pore size of the above-named predominant number of pores (i.e., the median of the pore size distribution) is in a range between 0.01 mm and 0.3 mm, preferably between 0.05 mm and 0.3 mm. In this embodiment, the absorption capacity of the body for the mixture is also still high, but overall somewhat lower than for the body with the bimodal pore size distribution. In the case of a body according to this exemplary embodiment of the present invention, the water absorption is at least 70% of the weight of the body, preferably at least 80% of the weight of the body. This means, for example, that if the body has a weight of 300 g, the water absorption capacity is at least 210 g, preferably at least 240 g. The high absorption capacity of the porous body also results in a long scenting time. In addition, in this exemplary embodiment the outer surface of the body is comparatively flat, which is due to the high proportion of small pores. In addition, during the molding of the green body, a relief can be pressed into the surface of the body so that a distinctive shape is produced.

In both of the above-named cases, the pore distribution for each group of pores can be approximated by a normal distribution.

In an exemplary embodiment, the body contains a sintered ceramic, preferably a silicate ceramic. Particularly preferably, the body is made up entirely of this sintered ceramic.

According to the present invention, the body is reusable, preferably up to 10,000 times. The filled mixture can be designed in such a way that it completely escapes from the body through evaporation. Alternatively, if the mixture does not completely escape from the body by evaporation, in order to be able to reuse the body the body can be heated by a heating device in order to completely remove any residue that may still be present in the pores. In this case, the removal is carried out by combustion. The heating temperature of the heating device is in the range between 200° C. and 1000° C., preferably between 650° C. and 900° C. Accordingly, the porous body is prepared by being baked at the temperature, for example over a period of 1 hour to 3 hours. The pore size distribution according to the present invention results in the heat of the heating device being able to penetrate ideally into the pores, so that the mixture can be combusted without residue. After thermal treatment, there are thus no longer any constituents of the previous “old” mixture left that would impair the quality and odor of the new filling After thermal treatment, the porous body can be refilled with a desired mixture according to the present invention.

The heating device can be constructed analogously to a conventional sintering furnace, which has a (re)sealable heating chamber. It can have a vent for any gas that may be produced, such that a vent flap may also be temporarily closed, and/or an air supply device.

The porous body may assume any three-dimensional external shape. This means that the outer surfaces of the body (disregarding the unevenness of the surface due to porosity) form the external shape. This shape can be produced by pressing or casting the green body before sintering or by appropriate post-processing (such as cutting and grinding) after the sintering. For example, the body may have a cuboid, cube, pyramid, cylinder, sphere, or plate shape. Any irregular shapes, shapes derived from the above shapes, and combinations of the above shapes can also be realized. The body can thus be adapted to the location of use, for example a housing of a dispenser. Because it is self-supporting, the porous body can be used either without a housing or with a mount or housing. When used without a mount or housing, the porous body can be placed on a support (for example, made of glass, stone, ceramic, chemically resistant plastic (for example, polypropylene), or high-grade steel if necessary, and at any location in a room to bring about the scenting of that room.

As explained above, an essential element of the system for scenting rooms is the porous body. The above problem is therefore solved, in particular, by a body which is self-supporting after sintering and comprises a porous, ceramic and reusable material, the material of the body having an open porosity greater than 50% and either
having a bimodal pore size distribution, a first maximum in the pore size distribution for the first group of pores being in the range between 1 mm and 3 mm and a second maximum in the pore size distribution for the second group of pores being in the range between 0.01 mm and 0.3 mm,
or a median of a pore size distribution being in the range between 0.01 mm and 0.3 mm.

In order to achieve the pore size distribution according to the present invention and thus a high absorption capacity of the body, the body is produced according to the method explained below. In particular, the method according to the present invention has the following steps:
Production of a mixture of unfired clay mineral, water, and fine wood dust and, if appropriate, a proportion of wood chips, and
Sintering the mixture at a temperature between 1000° C. and 1300° C. for a period of time in the range of 3 to 10 hours,

the mixture being shaped before sintering, for example by pressing, and/or the body being brought into the desired shape after sintering, for example by cutting and/or grinding.

Here, clay mineral is understood to mean a layered silicate and a silicate with a grain size <2 μm, and comprises at least one silicate of the group comprising two-layer silicates, three-layer silicates, layered silicates with mixed layers, clay minerals with a fibrous structure, and non-crystalline silicates. Two-layer silicates include kaolin and serpentine minerals such as kaolinite, dickite, halloysite, and antigorite, lizardite, and chrysotile. Three-layer silicates include pyrophyllite and talc, minerals of the mica group such as muscovite, of the smectite group such as montmorillonite, beidellite and saponite, of the hydro-mica group such as illite and glauconite, and in addition vermiculites and chlorites. The mixed-layer structures include the widely distributed irregular muscovite-montmorillonite mixed layers (also illite-smectite mixed layers). The clay minerals with fibrous structure are sepiolite and palygorskite. The non-crystalline silicates further include allophane and imogolite. Clay minerals are understood to be silica sand mixtures whose composition contains predominantly SiO2 and a mass fraction of up to 20% Al2O3 and up to 10% CaO. Small amounts of one or more compounds of the group containing TiO2, Fe2O3, MgO, K2O, and Na2O may also be present. A clay mineral is an inexpensive mineral raw material and is available in large quantities for many areas of industrial use.

The wood chips are preferably softwood chips, i.e., wood chips made of fir and/or pine wood. They are also called softwood pig meal because they are also used in animal husbandry as bedding. The wood chips are also called untreated softwood sawdust and are debarked, technically dried, and heat-treated. The softwood chips used for this application have an average chip size that ranges from 1 mm to 5 mm, preferably from 1 mm to 4 mm.

The fine wood dust is, for example, beech fine dust, which is also known as beech food smoking dust (e.g., from Thomsen Räucherspäne Räucherholz GmbH & Co. KG). The fine wood dust has a grain size of up to 0.02 mm.

To produce the mixture, the constituents are added in a weight ratio of unfired clay mineral to (fine wood dust and optionally a proportion of wood chips) to water in the mixture before sintering 1:(0.45 to 0.8):(0.9 to 1.2) and homogenized. Preferably, the weight ratio of unfired clay mineral to (fine wood dust and optionally a proportion of wood chips) to water in the mixture is 1:(0.5 to 0.75):(1 to 1.2). The resulting mixture is a heterogeneous mixture, which can be called a suspension or a mixture through the addition of water. Here, the above-indicated compositions of the mixture are understood such that the proportion of fine wood dust and wood chips is summed. If only fine wood dust is used, the weight ratio fraction of 0.45 to 0.8 (preferably 0.5 to 0.75) consists entirely of fine wood dust. If wood chips are also used, then part of this consists of the wood chips described above, and correspondingly less of the fine wood dust is present in the mixture. To achieve the bimodal pore size distribution described above, the weight ratio of wood chips to fine wood dust in the mixture is preferably 1:(1.2 to 2). For mixing, for example over a period of 5 minutes to 1 hour, preferably from 10 minutes to 45 minutes, a paddle kneader or a Z-arm mixer can be used.

The mixture is then sintered at a temperature between 1000° C. and 1300° C., preferably between 1100° C. and 1200° C., for a period in the range of 3 to 10 hours, preferably 4 to 7 hours, in air under normal pressure.

Before sintering, the mixture is shaped, for example by pressing or casting. For example, a ram pressing process, a pot pressing process or a horizontal pot pressing process is used. This produces green bodies, which, however, change again in size and/or shape as a result of sintering. In an exemplary embodiment, a relief structure can be introduced into the surface of the body, for example an upper surface, during pressing by a punch. This relief structure may have an indentation (score, groove) and/or raised part in the form of an image, characters in the form of letters and numbers, or the like. The indentation may preferably extend to a depth of 1.5 cm from the surface of the body. The relief structure is used, for example, for marking and decorating the body, and the relief structure is particularly easy to introduce into a green body containing only fine wood dust in the ratio indicated above, or at most a small amount of wood chips.

After sintering, the body can be brought into the desired shape and/or size by cutting and/or grinding, for example. The shape and/or size of the body is adapted to the intended use and the duration of the scenting.

The mixture with the at least one fragrance is made up of a plurality of constituents that exhibit different behavior with respect to escape from the porous body.

In this context, fragrances are any form of organic natural, nature-identical and synthetic fragrances. The term includes essential oils with all their constituents, especially terpenes and, from this group, in particular alcohol, glycoside, ether, aldehyde, ketone, carboxylic acid and ester terpenes. Also included are, in particular, other members of the class of aromatics, esters, alkyl pyrazines, aldehydes and ketones.

In a preferred embodiment, the mixture is a composition made up essentially of fragrances in the form of top notes and heart notes. Preferably, at least a portion of 80% by weight, preferably a portion of at least 90% by weight, of the mixture consists of top notes and heart notes. Here, top notes are components of the mixture that volatilize very quickly. Heart notes volatilize more slowly and form the “soul” of the fragrance produced by the blend. So-called base notes, which take a very long time to volatilize, are present in the composition only in a small proportion (less than 20% by weight, preferably less than 10% by weight, of the mixture).

Examples of essential oils that can be used for top notes are essential oils of bergamot, blood orange, cananga, christmas rose, clementine, strawberry, peach, red berries, eucalyptus, ginger grass, grapefruit, immortelle, chamomile, spearmint, lime, marjoram, lemon balm, mint, myrtle, palmarosa, rosewood, rosemary, juniper, wintergreen, cedar leaves, cinnamon, cypress.

Examples of essential oils for heart notes are essential oils of anise, bay oil, bitter orange, cajeput, verbena, fennel, guaiac wood, honey, immortelle, iris, calamus, carrot, mountain pine, lavender, lemongrass, mace, mandarin, mimosa, musk, nutmeg, niaouli, petitgrain, peppermint, pepper, sage, sweet orange, tea tree, vetiver, hyssop, cedarwood, cypress.

Essential oils that can be used as base notes are essential oils of asafetida, balsam turpentine, bay oil, birch bark, Damascene rose, elemi, galbanum, geranium, ho leaves, ginger, jasmine, mastic pistachio, clary sage, musk, myrrh, neroli, clove, patchouli, sandalwood, tea tree, tonka beans, vanilla, frankincense, cedarwood, civet cat, ylang ylang.

At least one of the above notes or a plurality of the above notes may be used in each case.

According to the present invention, the mixture also has a low flash point that is less than or equal to 120° C., preferably less than or equal to 110° C., particularly preferably less than or equal to 100° C. The flash point is determined by the Pensky-Martens method (DIN 51758, EN 22719) at normal pressure (1013 hPa). Mixtures according to the present invention having the named low flash point are characterized by the fact that they evaporate over a certain, comparatively short period of time without leaving a residue, because they have a low vapor pressure, so that the porous body can be reused and the odor of a new filling of the porous body is not impaired by residual products from the previous filling. In addition, the entire porous volume of the body can be used when refilling using a different mixture.

In an exemplary embodiment, the flash point of the mixture is greater than 60° C. If the flash point is less than or equal to 60° C., mixtures are classified as flammable liquids of category 1 (extremely flammable), 2 (easily flammable) and 3 (flammable) according to EU Regulation 1272/2008/EC, and appropriate measures must be taken during transport, storage and use. This additional effort is to be avoided in the mixture for the system according to the present invention.

The mixture according to the present invention is made up of a large number of individual compounds that are contained in the mixture in different concentrations.

The system according to the present invention is particularly environmentally friendly, because the porous body can be reused and refilled. The low environmental impact of the system according to the present invention for scenting a room is also due to the fact that the absorption capacity of the porous body, as described above, is comparatively large, so that the porous body can deploy its scenting effect in the respective room over the comparatively long period of time indicated above.

In the system according to the present invention, a mixture dispenser (hereinafter referred to as dispenser for short) may additionally be provided that has a housing for situating the porous body of the system described above. In this case, the situation of the body is such that fragrances can escape into the air of the surrounding space.

In an exemplary embodiment, the housing is designed to allow replacement of the body and/or refilling of the body with a different mixture.

In an exemplary embodiment, the housing is formed in a can-like manner and the situation of the body in the housing is such that the mixture can evaporate and escape the surrounding space at least in an opened state. This means that the gaseous mixture mixes with the air of the room. For example, the housing may be formed as a hollow body (e.g., hollow cylinder, hollow cone, hollow cuboid, hollow cube), the ceramic body being accommodated in the cavity of the housing. For example, the housing may be made of high-grade steel, glass, stone, or ceramic, or a combination of one or more of these materials. To allow the mixture to escape from the dispenser, the housing may include, for example, permanent through-openings or a removable, rotatable and/or slidable cover. In an exemplary embodiment, the cover of the housing may have a first position in which the cover closes the housing and a second position in which the cover is rotated and/or displaced relative to the first position such that the housing is at least partially unclosed in the second position. In the first position, the housing is thus sealed and the gaseous, vaporized mixture cannot escape to the outside. In the second position, the sliding and/or rotation of the cover forms an opening through which the mixture can escape. In an exemplary embodiment, the cover is completely removed.

In the case of the realization of the housing with permanent through-openings (e.g., circular, elliptical and/or angular openings) in a top wall and/or a side wall of the housing, in which no rotatable and/or sliding cover is provided, the housing can be closed before use by a correspondingly airtight packaging (e.g., a plastic packaging).

It has already been stated above that, according to the present invention, a method for preparing the system can also be carried out in which the porous body, after use, is baked in a heating device at a temperature between 200° C. and 1000° C., preferably between 200° C. and 650° C., in order to completely remove the mixture from the body, as appropriate. Subsequently, the porous body can be refilled with a mixture so that this mixture is absorbed into the body. In this way, a reusability of the system is realized, such that the same or different mixtures can be used in succession. By completely removing the mixture, the new mixture can deploy an (its) odor without this odor being impaired by the previously used fragrances.

Further features, advantages, and possible applications of the present invention also result from the following description of exemplary embodiments of a system according to the present invention and the figures. In this connection, all the features described and/or graphically illustrated constitute, in themselves or in any combination, the subject matter of the present invention, also independently of their summarization in the claims or relations of dependence therein.

### BRIEF DESCRIPTION OF THE DRAWINGS

FIGS. 1 and 2 schematically show a first embodiment of a system according to the present invention in a perspective view from the side, with the cover (FIG. 1) and without cover (FIG. 2),

FIGS. 3 and 4 schematically show a second embodiment of a system according to the present invention in a perspective view from the side, with cover (FIG. 4) and without cover (FIG. 3),

FIGS. 5 to 8 schematically show a third embodiment of a system according to the present invention, FIG. 5 showing the body in a perspective view from the side, FIG. 6 showing the housing in a perspective view from the side, FIG. 7 showing the housing in a view from above, and FIG. 8 showing the housing in a perspective view from below,

FIGS. 9 and 10 schematically show the third embodiment in different variants of the housing design in a perspective view from the side, and

FIG. 11 schematically shows the body of a further exemplary embodiment of the system according to the present invention in a partial view from above.

### DETAILED DESCRIPTION OF THE PREFERRED EMBODIMENTS

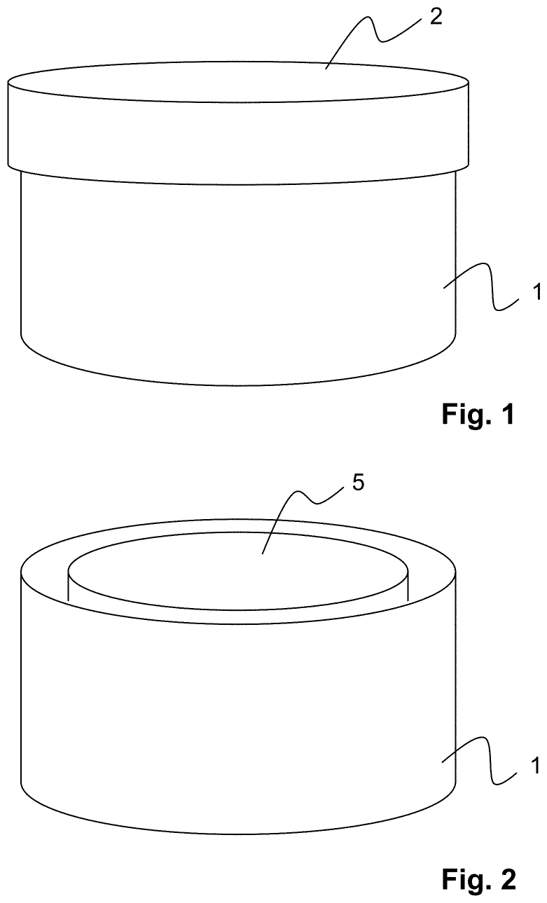

A first embodiment of a system according to the present invention is shown in FIGS. 1 and 2. The system has a hollow-cylindrical can-shaped housing 1, closed at the bottom, with a circular and removable cover 2 situated at the top, which closes off the interior of housing 1 from the surrounding environment. In the drawing of FIG. 2—with cover 2 removed—the interior of housing 1 can be seen. A cylindrical, self-supporting, solid and porous body 5 is situated there. Inside the body is situated the liquid mixture having at least one fragrance, which, when cover 2 is open, is released by evaporation into the surrounding space, thereby scenting it. When cover 2 is closed, the mixture cannot escape, because cover 2 seals housing 1 airtight with its surrounding rim. With cover 2 open, body 5 can also be removed in order to be freed of mixture residue by baking in a heating device and/or to be filled with a new, differently scented mixture.

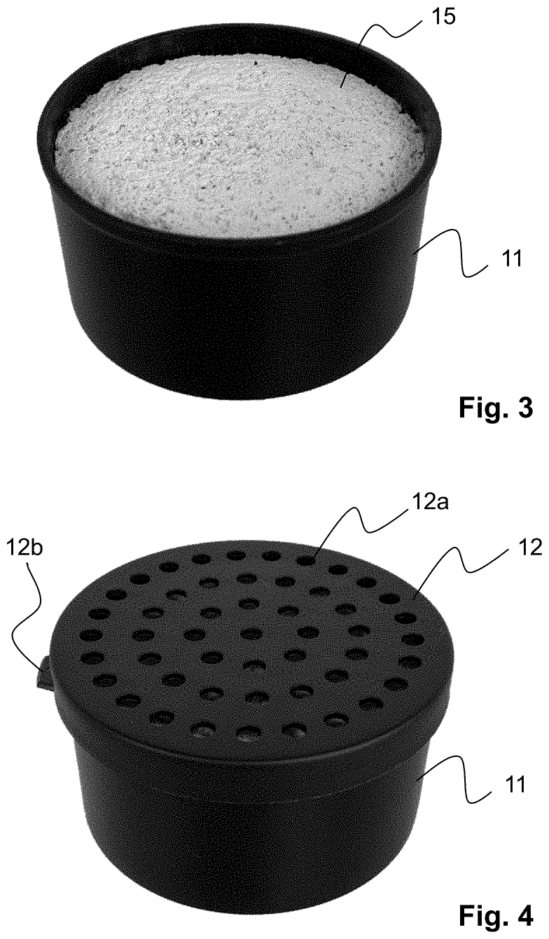

In the second exemplary embodiment shown in FIGS. 3 and 4, analogous to the first exemplary embodiment, a hollow-cylindrical housing 11 closed at the bottom is provided, which is closed by a removable cover 12 situated at the top. Inside housing 11 is situated cylindrical, solid, self-supporting and porous body 15 with the mixture accommodated in the body. Cover 12 has a plurality of circular through-openings 12a which, even when closed, allow the mixture to escape into the surrounding space for scenting. Cover 12 also has a tab 12b projecting in the radial direction (with respect to the axis extending through housing 11 or body 15) at the lower edge of the surrounding rim, which allows cover 12 to be easily grasped and pulled off.

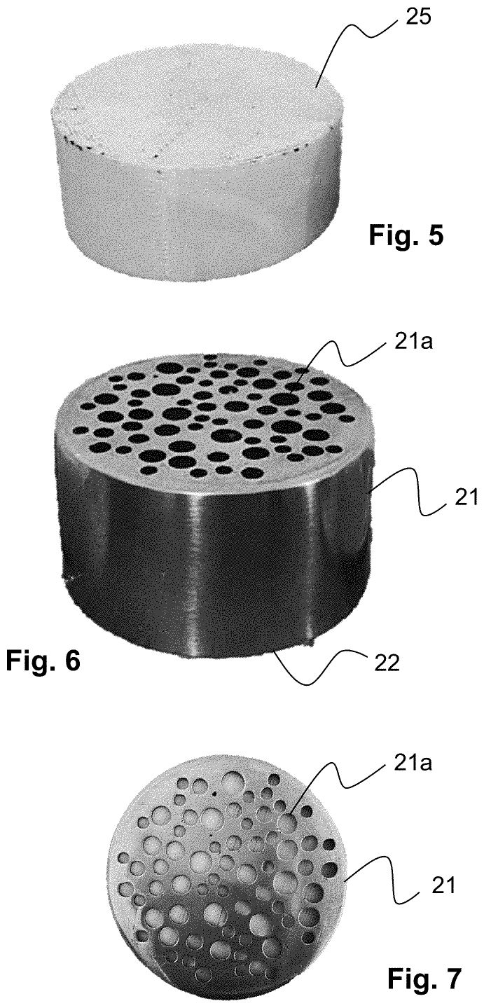

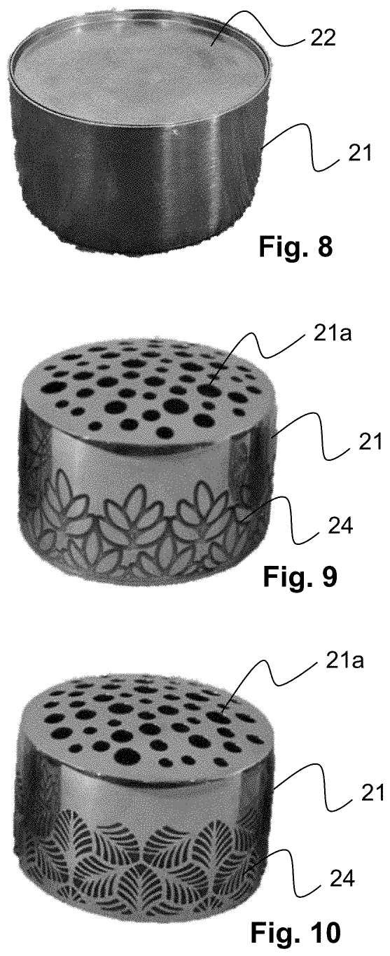

The third exemplary embodiment, shown in FIGS. 5 to 8, also has a hollow cylindrical housing 21 which is closed at the top, but has in its upper side a plurality of round or oval through-openings 21a which have different diameters (see top view in FIG. 7) and through which mixture evaporating from body 25 can escape into the surrounding space. In this embodiment, a cover 22 is situated on the underside, which closes housing 21 tightly and also in airtight fashion on this side, but can be removed in order to remove body 25. The self-supporting, ceramic, solid and porous body 25 is shown in FIG. 5 without housing 21. Body 25 is situated inside housing 21, as shown in FIG. 7, because the upper side of the body is visible through openings 21a. FIGS. 9 and 10 show two variants of the third exemplary embodiment with various decorations situated on the jacket surface of housing 21. These may be realized as engravings, colored printing, or the like.

In the above exemplary embodiments, the jacket surface may additionally or alternatively be provided with through-openings for the escape of the mixture.

The housing 1, 11, 21 of the above-explained exemplary embodiments of a system according to the present invention is made, for example, of high-grade steel or polypropylene.

The cylindrical porous body 5, 15, 25 for accommodating the mixture can be produced, for example, according to the following method. First, a mixture is produced of 1 kg of clay mineral (e.g., with the composition and mass fraction SiO2 73.9%, TiO2 0.7%, Al2O3 17.3%, Fe2O3 0.6%, CaO 6.0%, MgO 0.2%, K2O 1.2% and Na2O 0.2%), 250 g of softwood chips (pig meal) and 300 g to 500 g of beech fine dust, both, e.g., from the company Thomsen Räucherspäne Räucherholz GmbH & Co. KG, and 1 to 1.1 kg of water, using a Z-arm mixer. The ingredients are mixed for approximately 20 minutes. Then cylindrical green bodies are produced from the mixture using a pot press. These green bodies are then sintered at a temperature of 1150° C. to 1200° C. for 4 to 7 hours in air under normal pressure. If necessary, the sintered bodies can then be reshaped by grinding or cutting until the respective sintered body has the required dimensions and/or shape.

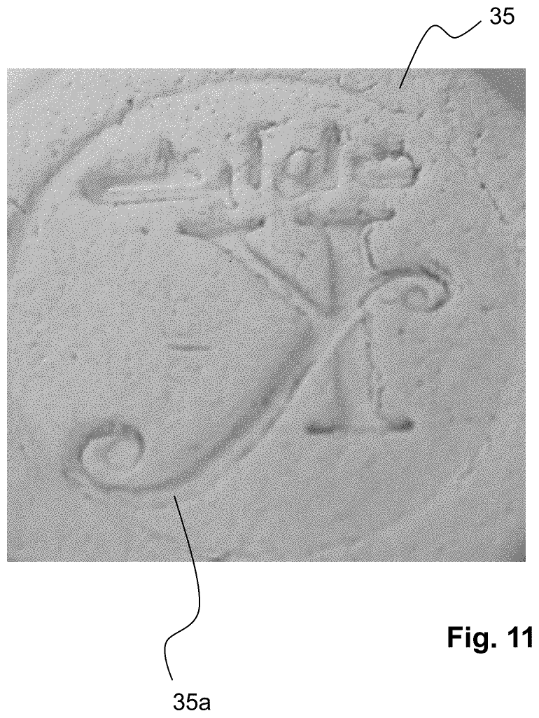

In an exemplary embodiment, shown in FIG. 11, during production of the green body a relief structure can be pressed into the surface of the body in a press (e.g., a pot press) using a suitably shaped punch. FIG. 11 shows an example of a body 35 for the production of which only beech fine dust was used (no softwood chips), with a relief structure 35a in the form of a depression consisting of various characters in the surface of body 35. The depression extends a few millimeters into the surface of body 35. The relief structure is also retained during sintering, but its dimensions may change during sintering, in a manner analogous to the body.

After the finished body has been placed in a dispenser with a housing 21 according to FIGS. 5 to 8, a certain predetermined amount of liquid mixture (e.g. 50 g) having at least one fragrance is further added to the body so that the mixture is absorbed into the body.

Housing 21 can then be closed at the bottom with cover 22. The completed system is then packaged in an airtight manner so that the mixture having at least one fragrance cannot escape before its intended use.

An example of a mixture containing a number of fragrances is a “Fresh Cotton” mixture. This blend has a flash point of 91° C. (at normal pressure) and is therefore very suitable for use in scenting a room and for use with the porous body described above. The mixture contains a relatively high proportion of acetates, which are cyclic, aromatic and aliphatic with at least 6 C-atoms.

| | | Portion |
|---|---|---|
| CAS no. | Name of the component | (wt %) |
| 140-11-4 | BENZYL ACETATE | 10-25 |
| 32210-23-4 | 4-tert-Butylcyclohexyl acetate | 10-25 |
| 58430-94-7 | 3,5,5-Trimethylhexyl acetate | 10-25 |
| 115-95-7 | Linalyl acetate | 5-10 |
| 142-92-7 | N-HEXYL ACETATE | 1-10 |
| 18479-58-8 | 2,6-DIMETHYL-7-OCTEN-2-OL | 1-5 |
| 110-98-5 | 1,1′-OXYDIPROPANOL | 1-5 |
| 112-31-2 | 1-DECANAL | 1-5 |
| 88-41-5 | 2-TERTIARY-BUTYLCYCLOHEXYL | 1-5 |
| | ACETATE | |
| 79-77-6 | (E)-4-(2,6,6-trimethylcyclohex-1-en-l-yl)- | 1-5 |
| | but-3-en-2-one | |
| 17511-60-3 | TRICYCLO DECENYL PROPIONATE | 1-5 |
| 2500-83-6 | TRICYCLO DECENYL ACETATE | 1-5 |
| 101-84-8 | DIPHENYL ETHER | 1-5 |
| 80-26-2 | P-MENTH-1-EN-8-YL ACETATE | 1-5 |
| 81786-73-4 | Acetyl diisoamylene, Z | 1-5 |
| 51566-62-2 | 3,7-dimethyloct-6-enenitrile | 0.1-5 |
| 706-14-9 | gamma-Decalactone | 0.1-5 |
| 110-41-8 | 2-Methylundecanal | 0.1-1 |
| 14765-30-1 | 2-(1-METHYLPROPYL)CYCLOHEXANONE | 0.1-1 |
| 470-82-6 | 1,8-Cineole | 0.1-1 |
| 67634-15-5 | A-DIMETHYL HYDROCINNAMALDEHYDE | 0.1-1 |
| 93-92-5 | METHYL PHENYL CARBINYL ACETATE | 0.1-1 |
| 1191-16-8 | 3-Methyl-2-butenyl acetate | 0.1-1 |
| 124-13-0 | 1-OCTANAL | 0.1-1 |
| 67634-00-8 | ISO-AMYL OXYACETIC | 0.1-1 |
| | ACID ALLYLESTER | |
| 85-91-6 | Methyl N-methylanthranilate | 0.1-1 |
| 137-03-1 | 2-N-HEPTYL CYCLOPENTANONE | 0.1-1 |
| 106-72-9 | 2,6-DIMETHYL-5-HEPTENAL | 0.1-1 |
| 93-04-9 | 2-Methoxynaphthalene | 0.1-1 |
| 141-13-9 | 2,6,10-Trimethyl-9-undecenal | 0.1-1 |
| 928-96-1 | BETA GAMMA HEXENOL EXTRA | 0.1-1 |
| 54464-57-2 | 7-ACETYL-(1,8)-OCTAHYDRO-1,1,6,7- | 0.1-1 |
| | TETRAMETHYLNAPTHALENE | |
| 18127-01-0 | 3-(4-TERTBUTYLPHENYL)PROPANAL | 0.1-1 |
| 19870-74-7 | CERDYL METHYL ETHER | 0.1-1 |
| 68039-49-6 | 2,4-DIMETHYLCYCLOHEXENE-3- | 0.1-1 |
| | CARBALDEHYDE | |
| 3681-71-8 | HEXENYL ACETATE CIS-3 | 0.1-1 |
| 70788-30-6 | 1-(2,2,6-trimethylcyclohexyl)hexan-3-ol | 0.1-1 |
| 106185-75-5 | 2-ETHYL-4-(2,2,3-TRIMETHYL-3- | 0.1-1 |
| | CYCLOPENTEN-1-YL)-2-BUTEN-1-OL | |
| 151-05-3 | APLHA, ALPHA-DIMETHYL | 0.1-1 |
| | PHENETHYL ACETATE | |
| 67634-20-2 | Tricyclodecenyl-8-isobutyrate | 0.1-1 |
| 13019-22-2 | 9-DECENOL | 0.1-1 |
| 99-49-0 | CARVONE | 0.1-1 |
| 68901-15-5 | ACETIC ACID, | 0.1-1 |
| | (CYCLOHEXYLOXY): | |
| | ALLYL ESTER | |
| 5986-55-0 | Patchouli alcohol | 0.1-1 |
| | (NATURAL CONSTITUENT) | |
| 93-08-3 | Methyl beta-naphthyl ketone | 0.1-1 |
| 4707-47-5 | Methyl atrarate | 0.1-1 |
| 87-44-5 | CARYOPHELENE CRUDE | 0.1-1 |
| 79-78-7 | ALLYL IONONE | 0.1-1 |
| 87731-18-8 | CYCLOOCTEN-4 AND 5-YL | 0.1-1 |
| | METHYL CARBONATE | |
| 110-27-0 | Isopropyl myristate | 0.1-1 |
| 122-03-2 | CUMIN ALDEHYDE | 0.1-1 |
| 65442-31-1 | 2-(2-METHYLPROPYL)-QUINOLINE | 0.1-1 |
| 2277-19-2 | CIS 6 NONENAL | 0.1-1 |
| 35854-86-5 | CIS 6 NONENOL | 0.1-1 |

Here, the above composition of the mixture is made such that all the ingredients together yield 100% by weight.

A quantity of 50 g of the above mixture “Fresh Cotton” will have completely escaped from the porous body in a 20 m2 room after two to three months. No residue is left.

Another mixture that can be used in the ceramic body described above is called “Morning Dew.” This mixture has the following composition and has a flash point that is between 100° C. and 120° C.

| | | Portion |
|---|---|---|
| CAS no. | Name of the component | (wt %) |
| 112-54-9 | DODECANAL | 0.1-1 |
| 67634-00-8 | ISO-AMYL OXYACETIC | 1-5 |
| | ACID ALLYLESTER | |
| 2705-87-5 | ALLYL CYCLOHEXANEPROPIONATE | 1-5 |
| 7493-74-5 | ACETIC ACID, PHENOXY ALLYL ESTER | 1-5 |
| 140-11-4 | BENZYL ACETATE | 1-5 |
| 122-63-4 | BENZYL N-PROPANOATE | small |
| | | portion |
| 99-49-0 | CARVONE | 0.1-1 |
| 2500-83-6 | TRICYCLO DECENYL ACETATE | small |
| | | portion |
| 103-95-7 | 2-METHYL-4- | 1-5 |
| | ISOPROPYLDIHYDROCINNAMALDEHYDE | |
| 68901-15-5 | ACETIC ACID, (CYCLOHEXYLOXY): | 0.1-1 |
| | ALLYL ESTER | |
| 18479-58-8 | 2,6-DIMETHYL-7-OCTEN-2-OL | 1-5 |
| 10094-34-5 | ALPHA,ALPHA-DIMETHYL | 1-5 |
| | PHENETHYL BUTYRATE | |
| 151-05-3 | APLHA, ALPHA-DIMETHYL | small |
| | PHENETHYL ACETATE | portion |
| 110-98-5 | 1,1′-OXYDIPROPANOL | small |
| | | portion |
| 105-95-3 | TRICANEDIOIC ACID: CYCLI | small |
| | | portion |
| 67634-15-5 | A-DIMETHYL | 1-5 |
| | HYDROCINNAMALDEHYDE | |
| 125109-85-5 | 3-(3-ISOPROPYLPHENYL)BUTANAL | small |
| | | portion |
| 63500-71-0 | 2-(2-METHYL PROPYL)-4-HYDROXY-4- | 1-5 |
| | METHYL TETRAHYDROPYRAN | |
| 67634-20-2 | Tricyclodecenyl-8-isobutyrate | 0.1-1 |
| 105-87-3 | 3,7-dimethyl-2,6-octadien-1-yl-acetate | 1-5 |
| 125-12-2 | EXO-1,7,7TRIMETHYLBICYCLO(2.2.1) | small |
| | HEPT-2YL ACETATE | portion |
| 1335-66-6 | 3-CYCLOHEXENE-1-CARBOXALDEHYDE, | 0.1-1 |
| | 2,4,6-TRIMETHYL | |
| 38285-49-3 | 4-ACETOXY-3- | small |
| | PENTYLTETRAHYDROPYRAN | portion |
| 706-14-9 | gamma-Decalactone | small |
| | | portion |
| 67633-96-9 | cis-3-hexenyl methyl carbonate | 0.1-1 |
| 2216-51-5 | DL-MENTHOL | small |
| | | portion |
| 141-12-8 | Neryl acetate | small |
| | | portion |
| 103-60-6 | 2-Phenoxyethyl isobutyrate | small |
| | | portion |
| 103-48-0 | 2-phenylethyl 2-methylpropanoate | small |
| | | portion |
| 93-92-5 | METHYL PHENYL CARBINYL ACETATE | small |
| | | portion |
| 78-69-3 | 3-OCTANOL, 3,7-DIMETHYL-Octanol | 10-25 |
| 81782-77-6 | 4-METHYL-3-DECEN-5-OL | 5-10 |
| 65443-14-3 | 2,2,5-Trimethyl-5-pentylcyclopentanone | small |
| | | portion |

The above composition of the mixture is made such that all the ingredients together yield 100% by weight.

A quantity of 50 g of the above fragrance mixture “Morning Dew” will have completely escaped from the porous body after two to three months in a 20 m2 room. No residue is left.

One of the mixtures shown above can be used in the above system. For use, the airtight packaging is removed and the dispenser is placed in a desired location in a room. There, the mixture slowly escapes through openings 21a of housing 21 into the air of the room and scents it. After a few weeks to 4 months, either the mixture has completely escaped from body 25 or residue is still contained in body 25. In the second case, the body is removed from housing 21 by removing the cover on the bottom of housing 21, and is tempered (baked) in air in a sintering furnace at a temperature between 650° C. and 900° C. to remove the residual amount of mixture remaining in body 25. Subsequently, body 25 can be refilled with a (different or the same) mixture. In the case of complete evaporation of the fragrance mixture from body 25, such baking is not necessary and the body can be refilled immediately. Housing 21 is then closed again by closing cover 22.

Because of the reusability of the system (about 10,000 times is possible), considerable amounts of packaging and containers can be saved which otherwise would have been discarded after each use of a predetermined amount of fragrance. Therefore, the system according to the present invention contributes to environmental protection.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A system for scenting rooms, comprising:
a solid body, and
a liquid mixture that contains at least one fragrance,
the solid body being configured to receive and allow evaporation of the liquid mixture,
the solid body comprising a porous, ceramic, and reusable material, with the material of the solid body having an open porosity that is greater than 50%,
the liquid mixture having a flash point that is equal to or below 120° C.

2. The system according to claim 1, wherein the ceramic material has a bimodal pore size distribution.

3. The system according to claim 2, wherein a first maximum in the pore size distribution for a first group of pores is in a range between 1 mm and 3 mm, and a second maximum in the pore size distribution for a second group of pores is in a range between 0.01 mm and 0.3 mm.

4. The system according to claim 1, wherein a median of a pore size distribution is in a range between 0.01 mm and 0.3 mm.

5. The system according to claim 1, wherein the liquid mixture forms a composition made up essentially of top notes and heart notes.

6. The system according to claim 1, wherein the solid body is configured to be heated by a heating device for a complete removal of the liquid mixture with a heating temperature being in a range between 200° C. and 1000° C.

7. A body configured to receive and allow an evaporation of a liquid mixture that contains at least one fragrance that is usable in the system according to claim 1,
the body comprising a porous, ceramic, and reusable material,
the material of the body having an open porosity that is greater than 50% and
either having a bimodal pore size distribution, a first maximum in the pore size distribution for a first group of pores being in a range between 1 mm and 3 mm, and a second maximum in the pore size distribution for a second group of pores being in a range between 0.01 mm and 0.3 mm, or
a median of a pore size distribution being in a range between 0.01 mm and 0.3 mm.

8. The body according to claim 7, wherein the ceramic material includes a silicate ceramic.

9. The body according to claim 7, wherein the body has on a surface thereof, a relief structure having at least one of a recess or a raised part.

10. The body according to claim 9, wherein the recess or raised part has an elevation differential of up to 1.5 cm relative to the surface of the body.

11. A method for producing the body according to claim 7, comprising the following steps:
producing a mixture of unfired clay mineral, water, and fine wood dust, and, optionally, a proportion of wood chips, and
sintering the mixture at a temperature between 1000° C. and 1300° C. for a period of time of 3 to 10 hours,
at least one of the mixture being shaped before sintering, or the body being brought into a desired shape after sintering.

12. The method according to claim 11, wherein the mixture is shaped before sintering by pressing or casting.

13. The method according to claim 11, wherein the mixture is shaped after sintering by at least one of cutting or grinding.

14. The method according to claim 11, wherein a weight ratio of unfired clay mineral, to fine wood dust and optionally a proportion of wood chips, to water in the mixture is 1:(0.45 to 0.8):(0.9 to 1.2).

15. The method according to claim 11, wherein a weight ratio of wood chips to fine wood dust in the mixture is 1:(1.2 to 2), if wood chips are used.

16. The method according to claim 11, wherein when the body is pressed before the sintering, a relief structure is pressed into a surface of the body by a punch.

17. The system according to claim 1, additionally comprising a dispenser having a housing configured to receive the solid body, the housing having a form of a can, and a receipt of the solid body in the housing taking place in such a way that, at least in an open state of the housing, the housing is configured to allow the liquid mixture to evaporate and escape into a surrounding environment.

18. A method for preparing the system according to claim 1, wherein after use, the solid body is baked in a heating device at a temperature between 200° C. and 1000° C.

19. The method according to claim 18, wherein the solid body is baked in the heating device at a temperature between 200° C. and 650° C.

20. The method according to claim 18, wherein after baking, the solid body is refilled with a liquid mixture such that the liquid mixture is absorbed into the solid body.
